(19) United States
(12) Patent Application Publication
(10) Pub. No.: US 2011/0021928 A1
(43) Pub. Date: Jan. 27, 2011

(54) METHODS AND SYSTEM OF DETERMINING CARDIO-RESPIRATORY PARAMETERS

(75) Inventors: Laurent Giovangrandi, Palo Alto, CA (US); Omer T. Inan, Palo Alto, CA (US); Keya R. Pandia, Stanford, CA (US)

Correspondence Address: Stanford University, 1705 EL CAMINO REAL, PALO ALTO, CA, 94306, US

(73) Assignee: The Boards of Trustees of the Leland Stanford Junior University, Palo Alto, CA (US)

(21) Appl. No.: 12/836,572

(22) Filed: Jul. 14, 2010

Related U.S. Application Data
(60) Provisional application No. 61/227,898, filed on Jul. 23, 2009.

(51) Int. Cl. A61B 5/0205

(52) U.S. Cl. 600/484

(57) ABSTRACT

Embodiments of the present invention provide noninvasive methods and systems of determining and monitoring an individual's respiration pattern, respiration rate, other cardio-respiratory parameters or variations thereof.

### RELATED APPLICATION

This application claims priority and other benefits from U.S. Provisional Patent Application Ser. No. 61/227,898, filed Jul. 23, 2009, entitled “Respiration monitor determining respiration with heart beat information”. Its entire content is specifically incorporated herein by reference.

### TECHNICAL FIELD OF THE INVENTION

The present invention relates to the field of noninvasive physiologic monitoring, in particular to methods and systems for determining an individual's cardio-respiratory parameters, including respiration patterns and rate, and/or variations in patterns and rates.

### BACKGROUND

The respiratory and cardiovascular systems work closely together to ensure that the oxygen demands of the body are adequately met. Regulation of these processes is supported by the autonomic nervous system via sympathetic and parasympathetic nervous control.

Respiratory disorders (e.g., chronic obstructive pulmonary disease/COPD, sleep apnea), resulting in an inadequate supply of oxygen (and removal of carbon dioxide), can lead to severe consequences. Conversely, many non-respiratory disorders lead to respiratory dysfunctions (e.g., cardiac heart failure). Proper diagnosis and continuous monitoring of individuals who have developed or who are at risk of developing such disorders is mandatory in order to avert serious, often life-threatening consequences.

Current methods to measure an individual's key respiratory parameters such as respiratory pattern, rate and volume typically require encumbering hardware, obtrusive methods of application, or utilize data-driven classification criteria which might not provide a consistently accurate representation of reality due to inherent limitations (Amit et al., 2009) and don't easily lend themselves to ambulatory, continuous monitoring that can, if needed, be carried out by the individual himself in a home environment.

The present invention addresses the inadequacies of these methods. It provides the ability for noninvasive, ambulatory and reliable continuous monitoring of an individual's respiration pattern and allows for determining cardio-respiratory parameters in or outside of the individual's home environment.

### SUMMARY

Embodiments of the present invention provide noninvasive methods and systems of determining and monitoring an individual's respiration pattern and rate by computationally processing single or combined respiration-dependent parameters to indicate the presence, development or absence of a respiratory, cardiac or neurological (syncope) disorder. In accordance with the various embodiments of the present invention, an individual's respiration can, thus, be monitored in an ambulatory and continuous fashion in or outside of the home environment.

In one particular aspect of the present invention, a respiratory screening system is provided for gathering respiration-dependent parameters in an individual via a sensor and computationally processing these parameters independently as well as in combination with each other using specialized algorithms to determine respiratory function and respiration rate and to provide at least one output function to indicate the presence, development or absence of a respiratory, cardiac or neurological (syncope) disorder. In a further aspect of the invention, methods for respiratory screening are provided for gathering respiration-dependent parameters in an individual via a sensor and computationally processing those parameters independently as well as in combination with each other using specialized algorithms to determine respiratory function and respiration rate and to provide at least one output function to indicate the presence, development or absence of a respiratory, cardiac or neurological (syncope) disorder. Optionally, the output function can include a benchmark signal from an alternative respiratory screening device or method as reference.

In one embodiment of the invention, a single, miniature and chest-worn accelerometer is utilized to capture a multitude of respiration-dependent parameters including chest wall motion, heart sounds attenuation (S1 and/or S2), S1-S2 interval and S1-S1 interval for independent or combined computational analysis to determine respiratory function and respiration rate and to indicate the presence, development or absence of a respiratory, cardiac or neurological (syncope) disorder.

Advantageously, particular combinations of respiration-dependent parameters can determine an individual's respiration rate with high accuracy and robustness for various postures as well as states of motion and independent of data-driven categorization criteria or training set requirements.

The above summary is not intended to include all features and aspects of the present invention nor does it imply that the invention must include all features and aspects discussed in this summary.

### INCORPORATION BY REFERENCE

All publications mentioned in this specification are herein incorporated by reference to the same extent as if each individual publication was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates an example set-up for determining an individual's respiration rate or other respiration-related diagnostic information, in accordance with the various embodiments of the present invention. Using a sensor, such as a chest-worn accelerometer, independent and mutually validating signal components are captured and evaluated for the determination of the individual's respiration-related diagnostic information such as respiration rate.

FIG. 2 illustrates an exemplary method for extraction of individual and combined respiration rates from raw acceleration signals recorded from an individual, in accordance with embodiments of the present invention. Various traces are extracted from the recorded signals and further processed to yield individual respiration rates that can be combined to yield a final respiration rate. Alternatively, the traces can be combined first and the respiration rate then extracted.

FIG. 3 illustrates the respiration-induced variation of the S1-S2 interval. Based on its location in the respiratory cycle (represented by the numbers on the reference respiration trace), S2 is more or less delayed compared to S1. Typically, S2 delay is shorter during inspiration, and longer during expiration.

FIG. 4 illustrates a specialized algorithm for the robust computation of the S1-S2 interval variation from (heart) beat to (heart) beat, in accordance with one embodiment of the present invention. This example is shown with a scanning range of −100 ms to +100 ms by steps of 5 ms, but these values can be changed for improved resolution/processing speed. Note that the segmented beat should at least contain S2 complex, but typically would span both S1 and S2 complexes as shown in FIG. 3.

FIG. 5 illustrates the principle of the algorithm for the computation of an individual's S1-S2 interval variation described in FIG. 4. The top plot shows two consecutive beats aligned on S1. The middle row shows different compression/dilation (by ΔD milliseconds) of beat N (solid line), compared to beat N-1 (dotted line). Note how the two beats best match for a compression of around 10 ms. By using finer steps, the maximum correlation can be accurately related to a compression by 9 ms. The S1-S2 interval of beat N is thus 9 ms longer than the interval of beat N. By repeating this step for all consecutive beats, the variation of S1-S2 interval can be quantified over the entire recording. Note also that this method is independent of the shape of S1 or S2, and can typically accommodate small variations in waveforms as seen during the respiratory cycle (such as common S1 or S2 split).

FIG. 6 illustrates how an individual's respiration waveforms, as derived from the S1-S2 intervals, S1 amplitudes, S1-S1 intervals (RSA) and chest wall motion compares favorably with the respiration waveform obtained with a reference respiration belt (bottom trace), as shown by the similar periodicity during normal breathing, and lack of during breath hold.

FIGS. 7-1 through 7-4 illustrate respiration waveforms derived from the S1-S2 intervals, S1 amplitudes, S1-S1 intervals (RSA) and chest wall motion, for an individual in four different positions: supine (FIG. 7-1), prone (FIG. 7-2), on left side (FIG. 7-3) and on right side (FIG. 7-4). The respiration belt waveform is also shown as reference, as well as the raw acceleration trace (bottom trace, after baseline wander removal).

FIG. 8 illustrates a walking individual's respiration rate, as derived from the S1-S2 intervals, S1 amplitudes, S1-S1 intervals (RSA) and chest wall motion in comparison to the respiration belt reference. The respiration belt waveform is also shown as reference, as well as the raw acceleration trace (bottom trace, after baseline wander removal).

FIG. 9 illustrates a resting individual's respiration waveforms, as derived from the S1-S2 intervals, S1 amplitudes, S1-S1 intervals (RSA) and chest wall motion in comparison to the respiration belt reference. For this figure and the raw data from beat features (unevenly sampled) are shown in gray (jagged traces), while re-sampled and filtered traces are shown in black (smooth traces).

FIG. 10 shows a typical electrocardiogram (ECG) signal alongside with the raw and baseline-removed acceleration signals that were recorded from an individual wearing an accelerometer as sensor on his chest. Also shown is a reference respiration belt signal (‘respiration amplitude’), all acquired simultaneously from the same individual.

FIG. 11 illustrates, based on the raw signals from FIG. 10, the respiration waveforms, as derived from the S1-S2 interval, S1 amplitude, S1-S1 interval (RSA) and chest wall motion in comparison to the respiration belt reference (reference respiration).

FIG. 12 shows Bland-Altman plots for respiration rates derived from the four individual respiration-dependent parameters chest wall motion, S1 amplitudes, S1-S2 intervals and S1-S1 intervals (respiratory sinus arrhythmia, RSA), compared to the rate derived from a reference respiration belt, recorded over 23 individuals. The X-axis shows the mean respiration rate (respiration per minute, rpm) over a 15 seconds window, while the Y-axis shows the respiration rate difference between accelerometer and respiration belt; the lines defined by the crosses indicates the 95% confidence interval.

FIG. 13 shows a Bland-Altman plot for the averaged respiration rate from all four parameters in FIG. 12; the lines defined by the crosses indicate again the 95% confidence interval.

### DEFINITIONS

Unless defined otherwise, all technical and scientific terms used herein have the same meaning as commonly understood by a person of ordinary skill in the art to which this invention belongs. The following definitions are intended to also include their various grammatical forms, where applicable.

The terms “determining”, “measuring”, “evaluating”, and “assessing” are used interchangeably and may represent quantitative and/or qualitative as well as relative or absolute measurements.

The terms “breathing” and “respiration” are interchangeably used in the present application and represent the inhalative/exhalative process by which oxygen is delivered from the external environment via the lungs to the blood and the cells in exchange for carbon dioxide.

The term “cardio-respiratory parameters”, as used herein, encompasses parameters such as respiration patterns, respiration rate and variation of these; the term encompasses furthermore parameters such as heart rhythm, heart rate, heart rate variability (HRV), respiratory flow, and variation of these, as well as interactions between cardio-respiratory parameters, mediated or not by the autonomic nervous system, such as respiratory sinus arrhythmia.

The term “respiration rate” or “respiratory rate”, as used herein, represents the number of breaths an individual takes within a given time interval (typically per minute).

The term “trace”, as used herein, describes a series of points spaced in time either continuously (evenly sampled trace) or discontinuously (unevenly sampled trace). The terms “trace”, “pattern”, “waveform” or “signal” are used interchangeably.

The term “chest expansion trace”, as used herein, describes the signal related to the physical motion of the chest during an inspiratory/expiratory cycle.

The term “respiratory sinus arrhythmia trace” or “RSA trace”, as used herein, describes the signal related to the modulation of the heart rate throughout the respiratory cycle, as estimated by S1-S1 interval variations.

The term “S1-S2 interval trace”, as used herein, describes the signal related to the variation of the timing between the first (S1) and second (S2) heart sounds throughout the respiratory cycle.

The term “S1/S2 amplitude trace”, or “S1 amplitude trace”, as used interchangeably herein, refers to the signal related to the modulation of the amplitude or energy in the heart sounds throughout the respiratory cycle. It typically refers, but is not limited, to the amplitude of S1, the energy of S1, the maximum amplitude of either S1 or S2, the maximum energy of S1 or S2, total energy of S1 and S2, or ratios of any of these metrics.

The term “algorithm”, as used herein, describes a finite sequence of steps that is executed using an automated data processing device such as a computer.

### DETAILED DESCRIPTION

Embodiments of the present invention provide noninvasive methods and systems of determining and monitoring an individual's respiration rate by computationally processing single or combined respiration-dependent parameters to indicate the presence, development or absence of a respiratory, cardiac or neurological (syncope) disorder. In accordance with the various embodiments of the present invention, an individual's respiration can, thus, be monitored in an ambulatory and continuous fashion in or outside of the home environment.

In one particular aspect of the present invention, a respiratory screening system is provided for gathering respiration-dependent parameters in an individual via a sensor and computationally processing these parameters independently as well as in combination with each other using specialized algorithms to determine respiratory function and respiration rate and to provide at least one output function to indicate the presence, development or absence of a respiratory, cardiac or neurological (syncope) disorder. In a further aspect of the invention, methods for respiratory screening are provided for gathering respiration-dependent parameters in an individual via a sensor and computationally processing those parameters independently as well as in combination with each other using specialized algorithms to determine respiratory function and respiration rate and to provide at least one output function to indicate the presence, development or absence of a respiratory, cardiac or neurological (syncope) disorder. Optionally, the output function can include a benchmark signal from an alternative respiratory screening device or method as reference.

In one embodiment of the invention, a single, miniature and chest-worn accelerometer is utilized to capture a multitude of respiration-dependent parameters including chest wall motion, heart sounds (S1 and/or S2) amplitude or combination of, S1-S2 interval and S1-S1 interval for independent or combined computational analysis to determine respiratory function and respiration rate and to indicate the presence, development or absence of a respiratory, cardiac or neurological (syncope) disorder.

Advantageously, particular combinations of respiration-dependent parameters can determine an individual's respiration rate with high accuracy and robustness for various postures as well as states of motion and independent of data-driven categorization criteria or training set requirements.

### Respiratory Disorders and Importance of Respiratory Monitoring

Respiration is the process by which in human individuals and other mammals, via the sacs of the lungs, fresh oxygen is delivered from the external environment to the cells in exchange for carbon dioxide. The respiratory system works in concert with the circulatory system to carry those gases to and from the tissues. Typical respiration is defined in terms of rate, regularity, and volume.

Since respiratory as well as autonomic nervous system disorders can develop when the oxygen demands of the body are not adequately met, monitoring of individuals who have developed or who are at risk of developing such disorders is important in order to avert serious, life-threatening consequences.

Pulmonary hypertension is an increase in blood pressure in the pulmonary (lung) vasculature and can be arterial, venous, hypoxic or thromboembolic. Pulmonary arterial hypertension develops due to gradual tightening and remodelling of the blood vessels connected to and within the lungs, which leads to increased pulmonary vascular resistance as well as pressure and, so, to less effective pumping of blood through the lungs and possibly to progressive right heart failure. As the blood flow through the lungs decreases, it becomes harder and harder for the left side of the heart to pump sufficient oxygen-rich blood into the circulation, especially during physical activity. In addition, extensive pulmonary vascular remodeling can initiate episodes of pulmonary embolism with life-threatening obstruction of the pulmonary vasculature. In case of pulmonary venous hypertension, the left heart fails to pump oxygen-rich blood efficiently into the circulation without any obvious physical obstruction of the blood flow. In hypoxic or secondary pulmonary hypertension, chronic low blood oxygen (hypoxia) is believed to cause constriction of the pulmonary arteries leading to a similar pathophysiology as explained above with pulmonary arterial hypertension. In chronic thromboembolic pulmonary hypertension the pulmonary blood vessels get blocked or narrowed with blood clots leading to increased pulmonary vascular resistance and possible right heart failure.

Pulmonary edema is an accumulation of extravascular fluid in the lungs that impairs exchange of oxygen and carbon dioxide and severely affects respiration, eventually leading to respiratory failure and possibly life-threatening respiratory as well as cardiac arrest.

Chronic obstructive pulmonary disease (COPD) is among the world's leading causes of death, partly because its etiology originates from harmful particles or gases such as cigarette smoke, which trigger episodes of abnormally strong inflammatory response in the lungs, gradually leading to a narrowing of the airways.

Similary to COPD, asthma is a common chronic inflammatory disease of the airways characterized by airflow obstruction and sudden episodes of difficulties in breathing due to constriction of the muscles in the walls of the bronchioles. Unlike COPD, the airway obstruction in asthma is usually reversible, if treated.

Sleep apnea is a sleeping disorder that is characterized by short episodes of breathing interruptions. While sleep apnea presents no immediate health risk, it can lead over time to severe sleep deprivation, low blood oxygen, and possibly congestive heart failure, thus, urgently requiring reliable methods and systems to screen for respiration abnormalities, as described by embodiments of the present invention. Sleep apnea is typically divided into two classes based on the primary cause: obstructive sleep apnea (physical obstruction of airways), and central apnea (lack of respiratory drive). Sleep apnea is typically diagnosed in sleep centers. Screening with portable polysomnographic systems is also possible, although remains costly and cumbersome.

Autonomic Nervous System Disorders

As part of the peripheral nervous system, the autonomous nervous system controls functions below the level of consciousness through its sympathetic and parasympathetic branches, which typically function in opposition to each other. So does sympathetic activation lead to an increase of the heart's contractility and heart rate with increased cardiac output, while parasympathetic activation has the opposite effect. Arterial baroreceptors are specialized blood pressure sensors in the blood vessels and participate in the regulation of cardiac output and blood pressure by modulating sympathetic and parasympathetic tones (baroreflex). They have a fast response time and are active in the regulation of blood pressure during transition from lying to standing, or sitting to standing, for instance. As such, they play an important role in disorders such as orthostatic hypotension, where baroreflexes contribute to the rapid compensation of the blood pressure change following the posture change. Baroreceptors are also one component of the respiratory sinus arrhythmias.

Syncope is a frequently encountered disorder which can be attributed to cardiac sources (cardiogenic), neurological sources (neurogenic), or both (neurocardiogenic, also called vaso-vagal syncope). Cardiogenic syncope is typically attributed to sudden drop in cardiac output due to arrhythmias. In the case of neurogenic or vaso-vagal syncope, the episode reflects a disorder of the autonomic system. In all cases, syncope is characterized by a sudden and temporary loss of consciousness that often leads to falls with more serious consequences. Ambulatory and continuous respiration monitoring, as described by embodiments of the present invention, would enable the detection of precursors of syncope in individuals by detecting changes in the transfer function between the various respiration waveforms derived from chest motion and heart sounds and related to different physiological subsystems (respiratory, cardiovascular, and autonomous nervous system). Diagnosis of syncope is typically performed by long-term ECG recording (Holter or loop recorder), or tilt-table tests. Prediction of impending syncope is currently not available.

### Heart Sounds and Sound Intervals

The cardiac cycle consists of two phases, namely systole and diastole. During systole the heart muscle contracts in response to an endogenous electrical stimulus and pressure is built up within the ventricles of the heart for the subsequent expulsion of blood into the aorta and the pulmonary arteries, respectively. Diastole describes the time period after systole, when the ventricles relax and fill again with blood.

The first and second heart sounds, S1 and S2, correspond to the closing of the atrioventricular and ventricular outlet valves, respectively, and can be picked up via a stethoscope. S1 occurs at the beginning of the contraction period (systole) and is produced by the closure of the mitral and tricuspid valves, while S2 occurs at the end of systole and is produced by the closure of the aortic and pulmonary valves. The time interval between S1 and S2 marks the systolic phase of the cardiac cycle (ventricular contraction), while the interval between S2 and S1 of the following cardiac cycle corresponds to the diastolic phase (ventricular filling).

Respiration parameters can be derived from heart beat by heart beat (beat-to-beat) S1-S2 timing interval changes as well as inter-heartbeat intervals S1-S1 intervals, as explained supra.

### UTILITY OF THE INVENTION

Embodiments of the present invention provide noninvasive methods and systems of determining and monitoring an individual's respiration pattern and rate by computationally processing single or combined respiration-dependent parameters to indicate the presence, development or absence of a respiratory, cardiac or neurological (syncope) disorder. Using one or more sensors that an individual can easily attach and comfortably carry for an extended period of time, independent and mutually validating parameters related to that individual's respiration and cardio-respiratory dynamics are measured and evaluated heart beat by heart beat, offering a robust and accurate method to determine an individual's breathing pattern and rate in various postures and states of motion, and to provide information on the interplay between different physiological subsystems (respiratory, cardiovascular, and autonomous nervous system). In accordance with the various embodiments of the present invention, an individual's cardio-respiratory dynamics can, thus, be monitored in an ambulatory fashion or not, within a medical facility or not, in a continuous or discontinuous (replacement for spot checks) fashion, in or outside of the home environment.

Ambulatory and continuous respiration monitoring would be highly relevant for general physiological and cardio-respiratory monitoring from home, while resting or exercising, and is so far limited. Ambulatory and continuous respiration monitoring would, furthermore, be highly beneficial for sleep respiration monitoring, and for respiration monitoring at trauma and accident sites. In addition, ambulatory and continuous monitoring of changes in the transfer function between the various respiration waveforms derived from chest motion and heart sounds might greatly benefit the monitoring of some neurological disorders, such as the monitoring, diagnostic and prevention of neurogenic, neurocardiogenic or undiagnosed syncope.

Sleep apnea monitoring would also benefit from the cardio-respiratory monitoring offered by the present invention. In addition to providing multiple respiration signals with different physiological origin (chest expansion, cardiac, blood pressure, and autonomous nervous system), the same sensor (accelerometer) readily provides information on posture and motion, as well as heart rate. For instance, obstructive vs. central apnea can be separated on the basis of mechanical chest movement, as detected by the chest wall motion trace, as well as to a lesser extent by the posture at the time of the apneic event (obstructive apnea occurs predominantly in supine position, Oksenberg, 1997).

The determination of an individual's respiration rate is not easily accomplished and typically requires encumbering hardware and/or obtrusive methods of application, as it is the case with the currently used standard respiration belt (aka respiration monitor belt), impedance plethysmograph or flow sensor such as spirometers. Besides being obtrusive, these methods yield inaccurate and possibly misleading results due to motion artifacts, coughing or inherent computation-based limitations.

A typical respiration belt is strapped around an individual's chest and then inflated with air. The individual's respiration is then measured by monitoring the pressure that results from the expansion and contraction of the chest during breathing. Alternate belt technologies use inductive, piezoresistive or piezoelectric transduction to convert the mechanical movement of the chest expansion into an electrical signal. Respiration belts require a tight fit around the chest, uncomfortable for long-term use, in order to produce consistent signals.

An impedance plethysmograph measures the resistance of the chest and its modulation by the varying lung volume during respiration. Impedance plethysmography requires electrical contact to the skin, usually using sticky electrodes similar to ECG electrodes. A spirometer measures with an air flow sensor the amount and rate of air that is inhaled and exhaled by an individual. Accurate measurements require the individual to blow through a tube while the nostrils are pinched closed. Spirometers are typically not used for long-term, continuous measurement due to their obtrusiveness.

Computational approaches that are based on algorithms for pattern recognition and necessitate data-driven classification criteria (Amit et al., 2009), might not provide a consistently accurate measurement due to inherent limitations of the training process and assumption of cyclostationarity. Indeed, such approaches are very sensitive to morphology changes due to external factors, such as posture or motion.

Technical Details

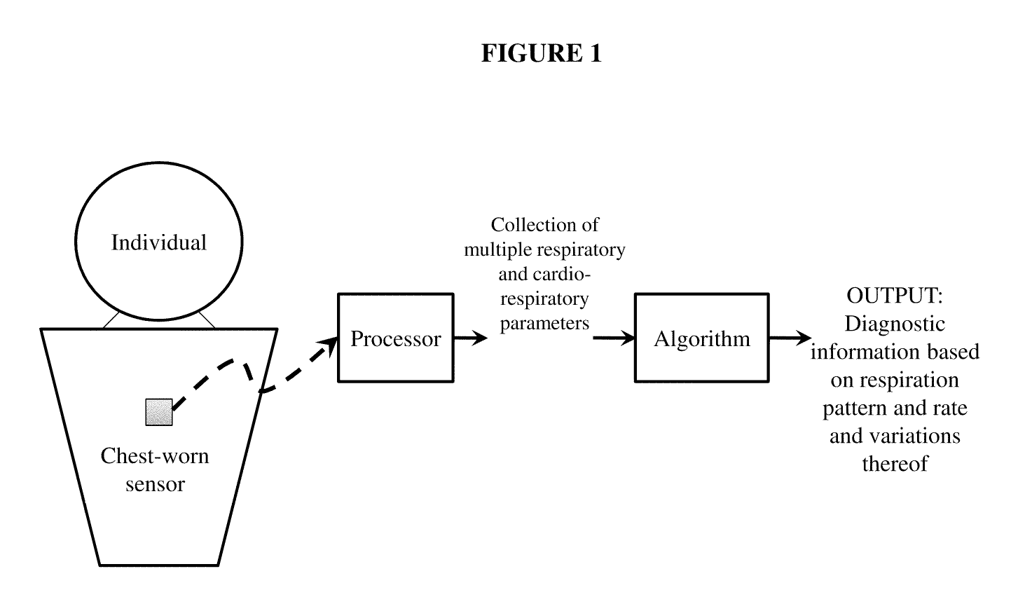

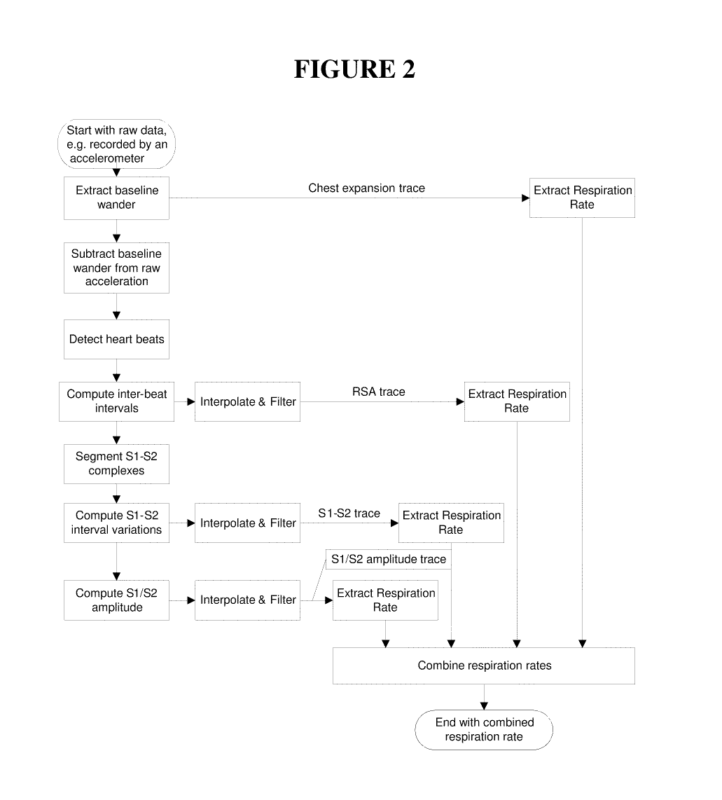

As schematically illustrated in FIGS. 1 and 2, in the various embodiments of the present invention four independent, mutually validating signal components are captured and evaluated that derive from a) the mechanical movement of the chest by expansion of the lungs and b) from the acoustic waves that are generated by the heart valves closure to determine an individual's respiration pattern and rate in a robust, reliable and accurate fashion, in various postures and states of motion and independent from data-driven categorization criteria or training set requirements.

Chest Wall Motion

A sensor, which is an accelerometer in one embodiment of the present invention, attached to an individual's chest, as depicted in FIG. 1, detects the mechanical motion of the chest wall in response to respiration, similar to a respiration belt, which only detects the expansion and contraction of the chest cavity. This motion is manifested in the form of a low-frequency (<1 Hz) baseline wander in the detected acceleration signal. From the baseline wander, a chest expansion trace is extracted, from which, in turn, the respiration rate is extracted.

Heart Sound Amplitude Variations

The propagation path of the higher frequency (>1 Hz) primary heart sounds to the chest is modulated through the respiration-dependent movements of the chest wall. This is due to the fact that the distance between the chest-worn sensor and the sound source varies directly with respiration-dependent chest wall motion, causing a change in the intensity of the signal detected by the sensor. The change in signal propagation path leads to variable signal attenuation, a modulation of power or amplitude, and is picked up by the sensor. In addition, the amplitude of S1 and S2 sounds are themselves modulated throughout the respiratory cycle as stroke volume and pressures are affected: higher pressure differences through the valves lead to faster and stronger impacts and decelerations at valve closure and thus louder sounds.

Several metrics heart sounds amplitude can be used to quantify the heart sound amplitudes and their modulation, including but not limited to, maximum absolute amplitude, peak-to-peak amplitude, energy of the whole beat (encompassing S1 and S2), of individual components (S1 or S2), or combination of (ratio of S1 and S2 amplitude or energy, to capture opposite changes during the respiratory cycle). These metrics, occurring at the beat locations in time (hence unevenly sampled), are then interpolated (resampled) and low-pass filtered to generate a respiration trace based on the S1/S2 amplitude, as shown in FIG. 2.

S1-S2 Timing Interval Variations (S1-S2 Intervals)

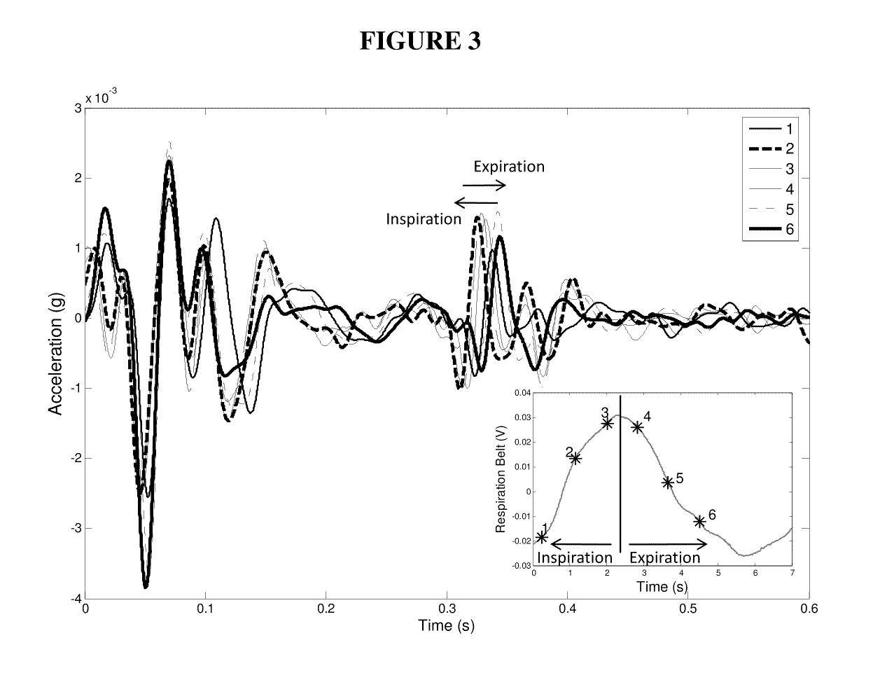

Respiration modulates the timing of the primary heart sounds S1 and S2 in subtle and indirect ways. Inspiration decreases pleural pressure, and applies pressure on the systemic venous and arterial system. Increased pressure on the venous system increases venous return and pre-load in the right ventricle. The longer filling time results in delayed S1. In the left ventricles, the smaller pre-load (due to interactions between the two ventricles) and higher after-load result in a shorter systole, and an earlier S2. These two effects result in a shortening of the S1-S2 interval during inspiration, and a widening during expiration. This effect is illustrated in FIG. 3. Similarly to amplitude metrics, the S1-S2 intervals (or variations of), occurring at the beat locations in time (hence unevenly sampled), can then be interpolated (re-sampled) and low-pass filtered to generate a respiration trace based on the S1-S2 intervals, as shown in FIG. 2.

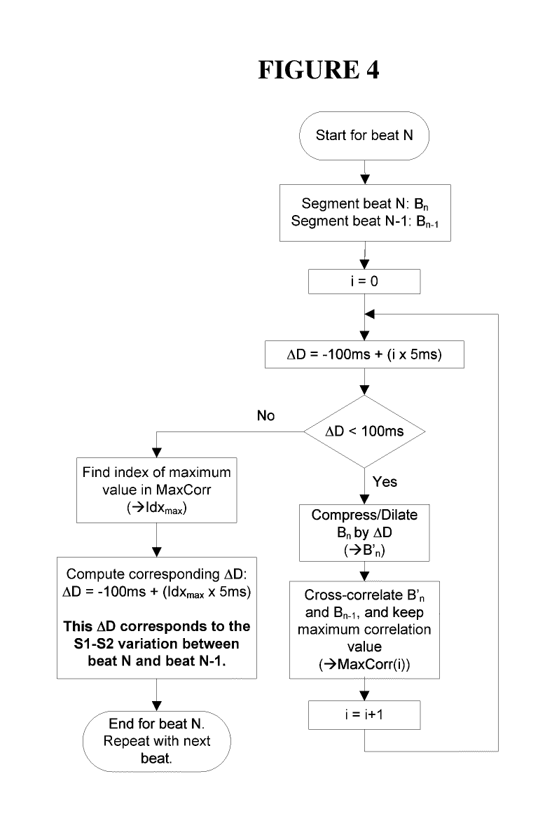

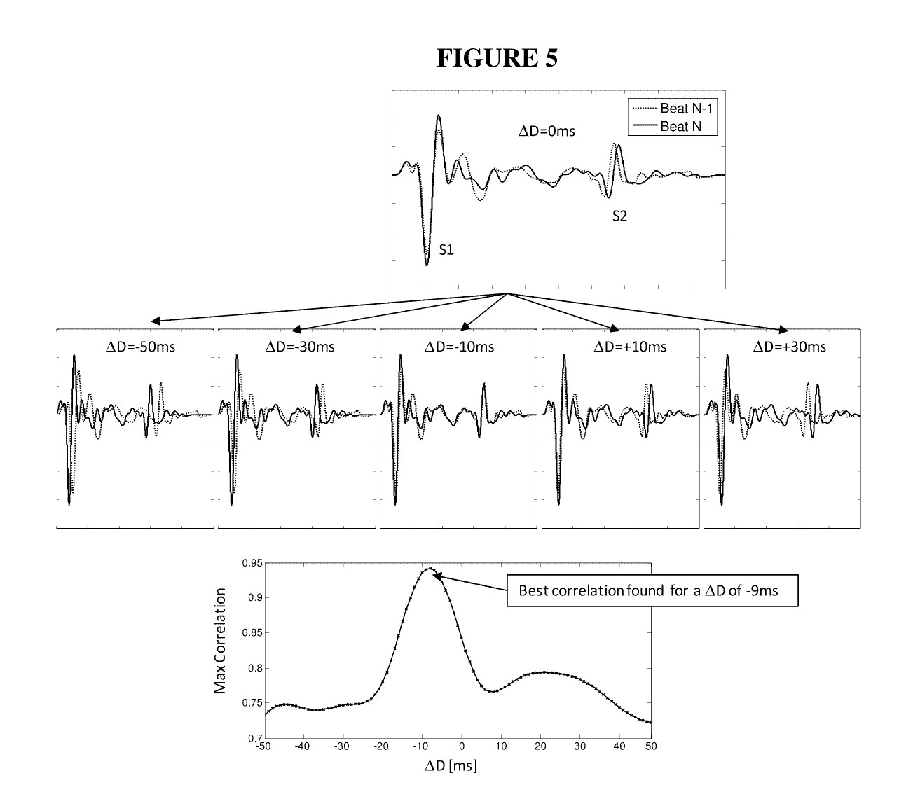

FIG. 4 presents an exemplary computation of an individual's S1-S2 interval variation from (heart) beat to (heart) beat using an algorithm that is further illustrated in FIG. 5. Robust estimation of the variation of the S1-S2 interval from one beat to the next can be obtained by finding out how many milliseconds the next beat has to be compressed or stretched to best match the previous one. For each couplets of beat, the maximum correlation values between the first beat and successive compressions or stretching of the second beat are computed, and the compression or stretching amount corresponding to the largest overall correlation value is taken as best estimate of the S1-S2 interval variation between these two beats. The compression and stretching are typically achieved by interpolation and re-sampling of the beat. Range and step size for the compression/stretching can be optimized for resolution or speed. Alternatively, metrics of similarity other than correlation (e.g., mean squared error, Euclidian distance) could be used to provide optimal performances. The process is repeated for all successive couplets of beats. Because the algorithm only compares successive beats, it is robust to change in morphology over time (as can occur with posture changes), as well as to morphology changes due to respiration-induced S1 and S2 splits. Also, since it compares entire beats (spanning both S1 and S2 sounds), it is robust to beat detection errors. For instance, if the beat detection triggers on S2 instead of S1, and assuming the windowing around the trigger point (fiducial) is large enough to include S1 and S2, accurate estimation of the S1-S2 interval variation can still occur. On the opposite, if S1 detection is highly accurate, then the correlation could be computed only on S2 (instead of the entire beat), or vice-versa, thus reducing computational load.

S1-S1 Inter-Beat Interval Variations (Respiratory Sinus Arrhythmia)

Respiratory sinus arrhythmia (RSA), one of the physiologic interactions between the respiratory and cardiovascular system, is a well-known phenomenon with complex, physiologic bases. It represents the synchronization of the heart rate variability (traditionally measured using ECG) and respiration. During inspiration, the RR intervals in the ECG are shortened, while they are prolonged during expiration. RSA has been used at occasions for estimating respiration. However, the presence and magnitude of RSA is subject-dependent, and is typically reduced in older subjects, or in subjects with cardiovascular diseases. Also, RSA is influenced by factors such as body position (posture), gender, sleep/wakefulness, level of fitness. Relying solely on RSA for respiration monitoring is thus unreliable. However, used in conjunction with other markers of respiration (such as proposed here), the presence, form or absence of RSA becomes valuable information. Detection of Cheyne-Stokes breathing, frequent in patients with congestive heart failure (CHF), is a good example: in this case, RSA does not vary with the breath-by-breath periodicity of normal breathing, but rather with the frequency of the hyperpnea episodes. Without another marker of respiration, RSA could mislead into a low breathing frequency reading. With an additional, independent marker (chest wall motion, S1/S2 amplitude, or S1-S2 interval), such rhythm can be identified. Periodic breathing—another form of slow breathing oscillations (cycle length: 25-100 s) often found in CHF patients, is another example where the relation (phase) between RSA and ventilation (e.g., chest motion) allows the identification of the dominant mechanisms (Pinna et al., 2000).

RSA is primarily mediated by the parasympathetic system, and is controlled by a range of central and peripheral vagal control mechanisms. As such, it has been for a long time regarded as an indicator of vagal tone. Central processes (respiratory drive) as well as peripheral mechanisms (chemoreceptors, baroreceptors, lung inflation) all contribute with relative strength at various points in the respiratory cycle: during inspiration, the central respiratory drive strongly attenuates vagal efferent signals, while the vagal efferent discharges are maximal during expiration (Grossman et al., 1993; Yasuma et al., 2004). Note that derivation of RSA from S1-S1 rather than R-R interval introduces a slight inaccuracy in the estimation of RSA due to the variation in the delay of S1 with respect to the R-wave throughout the breathing cycle (as mentioned above). However, this variation is typically small (milliseconds) compared to RSA (tens of milliseconds).

Respiration Traces

These four, above detailed signal components can be computationally evaluated as independent parameters or in combination with each other as mutually validating parameters, providing a robust and reliable method of determining the pattern and rate of respiration. Using specific algorithms (see again FIG. 5), respiration traces derived from S1-S1 intervals (respiratory sinus arrhythmia/RSA), S1 amplitudes and S1-S2 intervals are obtained by reconstructing continuous, evenly-sampled waveforms from the series of discrete samples obtained at every heart beat. Such reconstruction is achieved by cubic spline interpolation of the unevenly-sampled data and re-sampling at a fixed sampling frequency. The fourth trace, chest wall motion, is readily derived from the baseline wander of the raw acceleration.

The individual respiration traces derived from the various features, and in particular the relationship between these traces, can be used for diagnostic purposes. These relationships can be quantified by transfer functions. These transfer functions can be used to characterize the interplay between respiratory, cardiac and autonomic systems. On this basis, monitoring of these transfer functions over time may capture or provide early detection of impending events such as syncope or other autonomic system dysfunctions.

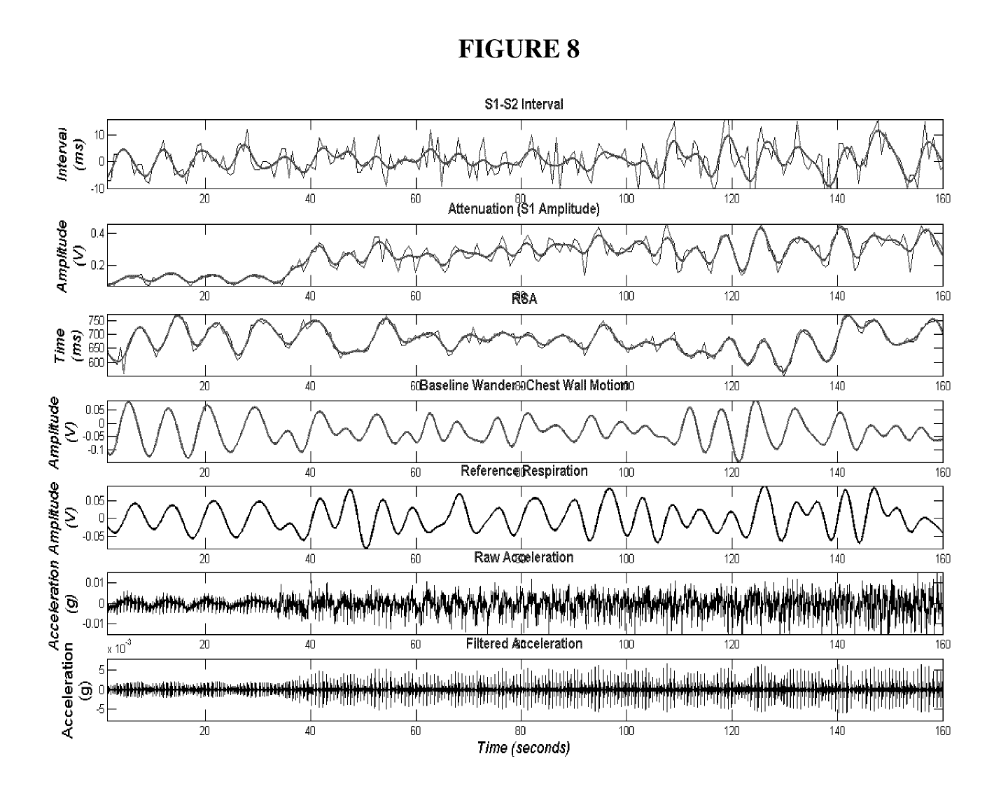

Various embodiments of the present invention demonstrate that the respiration waveforms, as derived from the described parameters in combination or in singularity, represent with fidelity the respiration effort measured by a respiration belt (see FIG. 8). While the various traces show some phase shift between each other (as expected from their various physiological origins), all traces accurately represent the oscillatory nature of the respiration, as well as the lack of oscillation during breath hold.

Respiration Rate

For all or a subset of these four respiration traces, a respiration rate is extracted using a frequency-domain technique (short-window Fourier transform). Alternatively, other methods of instantaneous frequency determination can be used (including, but not limited to, autocorrelation). Once respiration rates have been computed for one or more traces, a robust estimate can be derived by combining the rates. In the present embodiment, a simple averaging was used. However, more sophisticated data fusion approaches could be used, including weighted averages, where the weight could be determined as a function of the strength of the periodic signal (as measured by the relative energy in the respiration frequency range of the power spectrum), or using other information derived from the same accelerometer such as posture or motion. Indeed, if motion is detected, the chest wall motion trace is likely to be corrupted by motion artifact, and it thus can be attributed a lower weight in the weighted average. Similarly, if the individual is in prone position, the chest wall motion signal will have very low respiratory component and its weight can be lowered.

### Sensors Suitable for Embodiments of the Present Invention

The various embodiments of the present invention utilize at least one sensor that is placed on an individual's upper torso, preferably on the chest close to the sternum. Due to a natural variation in the mechanical axis of the heart across different individuals, the use of multiple sensors in a grid-like configuration to adequately spatially sample the chest area around the heart may be indicated, if optimal sensor placement is desired without manual intervention. Alternatively, manually fine tuning the sensor placement for each individual will also achieve a spatially optimum sensing location for each individual.

A multi-sensor approach might also be used to provide various types of information (emphasis on S1 or S2 through optimal localization), for redundancy, or to support various noise-cancelation schemes.

Accelerometers

An accelerometer is a device that measures, on contact, the acceleration of a surface via a sensing element. Typically, when the accelerometer is subjected to an acceleration, the movement of a proof mass is converted to electricity via piezoelectric, piezoresistive or capacitive transduction. Micromachined accelerometers are miniature accelerometers that can integrate multiple axis and typically contain conditioning circuitry for easy interfacing with standard electronics. They can be very small (less than 5×5×2 mm), lightweight and low-power. Chest-worn accelerometers have been shown to detect seismocardiogram (SCG) signals that contain indicators of the primary heart sounds S1 and S2.

Single-Axis Versus Multiple-Axis Accelerometers

The use of a single-axis accelerometer as a sensor in an embodiment of the present invention is sufficient to capture heart sounds. In embodiments of the invention, where postural and state of motion information should be captured as well, for example to determine an individual's sleeping position, in particular, when in the prone position, to detect motion as to influence a data fusion algorithm (as illustrated above), or to detect if an individual whose respiration rate is determined falls as a consequence of experiencing a syncope, the use of a three-axis accelerometer (e.g. ST Microelectronics LIS344ALH) is preferable. Due to the small amplitude of signals measured (in the milli-g range), accelerometers with low noise floor (<50 ng/sqrt(Hz)) are typically used.

Alternatives to Accelerometers

Laser (Doppler) Vibrometer

Laser vibrometers provide non-contact vibration measurements (vibration amplitude and frequency) of a surface using a laser beam directed at that surface. The use of a laser vibrometer may be preferable over an accelerometer, if a measurable entity is difficult to access or otherwise not suitable to contact vibration measurements (for instance, burn victims).

Stethoscope

A digital stethoscope (phonocardiograph) can be used to record heart sounds from which the three heart sound parameter described above (S1-S1 intervals, S1 amplitudes, and S1-S2 intervals) can be derived.

Microwave Radar

An electromagnetic, microwave radar emits electromagnetic waves that are scattered and partially reflected when they get into contact with a surface or an interface. In the case of Doppler radar, the frequency of the reflected waves is modulated according to the velocity of the surface or interface, thus providing information about its motion. Although not necessary, differentiating this motion signal would provide an acceleration signal similar to the one given by an accelerometer. Similarly to laser vibrometry, microwave radars do not require contact. However, they can also work through clothes, and are less sensitive to surface properties (reflectance).

### Systems Suitable for Embodiments of the Present Invention

Systems to determine an individual's cardio-respiratory parameters, including respiration patterns and rate, as contemplated, comprise a) a sensor that is placed such as to capture a) the mechanical movement of the individual's chest by expansion of the lungs and b) the acoustic waves that are generated by the individual's primary heart sounds S1 and S2. Such systems further comprise b) a data acquisition device to receive information from the sensor and to output the information to a c) processor, such as an external personal computer, for interpretation of the received information and graphical representation. The transfer of information from a) the sensor to b) the data acquisition device and/or c) the processor can be carried out in wired or wireless operation. In a current embodiment, an ST Microelectronics LIS344ALH 3-axis analog accelerometer was amplified and interfaced to a National Instruments acquisition card connected to a personal computer.

The processor is adapted such that the received information can be processed and interpreted individually or in combination using specialized algorithms to determine the individual's respiration rate. Apart from an individual's respiration pattern and rate, the processor is also adapted to measure heart rate based on heart sounds (S1-S1 interval, for instance), and to capture and quantify abnormal cardiac events (bradycardia, tachycardia, premature ventricular contractions, asystole).

All these functions could also be integrated into a miniature sensor patch based on a single sensor and a microcontroller (possibly wireless-enabled for data transmission), or even in an ASIC (Application-Specific Integrated Circuit). Data processing could be performed locally within the microcontroller, or on a secondary device in the form of a cell phone or a dedicated system, in which case the microcontroller or ASIC would send wirelessly the raw acceleration signals to the secondary device. Alternatively, the raw acceleration signals could be stored in memory either on the sensor patch or on the secondary device for further processing on a personal computer after downloading of the data.

In one particular embodiment, the processor estimates the variation in S1-S2 intervals between two consecutive heart beats by incrementally compressing and stretching them until they best match (highest correlation), yielding a dilation coefficient directly reflecting S1-S2 interval variation between the two consecutive beats, which is then used to generate a respiration rate.

Systems to determine an individual's cardio-respiratory parameters, including respiration patterns and rate, in accordance with embodiments of the present invention, may be used in an ambulatory or stationary fashion to continuously or discontinuously monitor an individual's respiration rate in his home or away from home. An exemplary application of such systems is the detection of sleep apnea where the system is utilized to quantify apneic events and to provide an indication of the type of the experienced apneic events (obstructive, central apnea), as extracted from the individual's respiration traces (each related to different physiological subsystems—respiratory, cardiovascular, autonomic nervous system), heart rate and its variability, posture and motion, all derived from data sensed by a single accelerometer.

Such systems may also be used for continuous monitoring of an individual's cardio-respiratory parameters, including respiration patterns and rate in a hospital, trauma center or other health care facility, and also during recreational physical activities such as running, bicycling and so forth.

### Specialized Algorithms

Algorithms were developed to extract the respiration waveform and respiration rate from respiration-dependent parameters independently as well as in combination with each other, particularly from a) chest wall motion, b) S1 amplitudes, c) S1-S1 intervals, also called inter-beat interval or respiratory sinus arrhythmia (RSA) and d) S1-S2 intervals, also called S1-S2 time intervals (a varying interval between the primary heart sounds) by capturing 1) variation over time of the time interval between S1 and S2 events and (2) variation over time of the sensed amplitude of an entire heart beat, of S1, of S2, or a combination of S1 and S2. Derived respiration waveforms and rates were compared to those obtained from a reference respiration belt and analyzed across individuals and recordings, as described earlier and illustrated in FIGS. 1-5.

For a personalized computational analysis of one or more respiration-dependent parameters, respiration detection algorithms can be tailored for every individual by differently weighting the response of each parameter in computing the respiration-rate. For instance, if a person does not have a strong RSA (because of age or some pre-existing cardiac disease), then the RSA-derived rate can be given a lower weight in a weighted average, so as to not introduce erroneous data in the average.

Similarly, the weight could be automatically determined in real-time as a function of the strength of the periodic signal (as measured by the relative energy in the respiration frequency range of the power spectrum), or using other information derived from the same accelerometer such as posture or motion. Indeed, if motion is detected, the chest wall motion trace is likely to be corrupted by motion artifacts, and it thus can be attributed a lower weight in the weighted average. For sleep monitoring, if the individual is in prone position, the chest wall motion signal will have very low respiratory component and its weight can be lowered.

### Independent or Combined Computational Analysis

The advantage of combining multiple parameters to assess respiration function and rate is especially pronounced for individuals who do not show a strong respiration dependence on any single individual parameter. The autonomic feedback loop, for instance, is known to weaken with age, statistically making RSA a poor index for respiration in the elderly population.

### Posture and State of Motion

Embodiments of the present invention address the unmet need for a respiration monitoring method and system that, among other advantages, can be used by an individual in an ambulatory setting, in an awake or unawake state, in motion, standing, sitting, lying for rest or sleeping, at home or outside of the home. Therefore, the methods and systems of the present invention were tested, in comparison to a reference, to ensure suitability and reliability at various postures or states of motion such as walking.

Posture

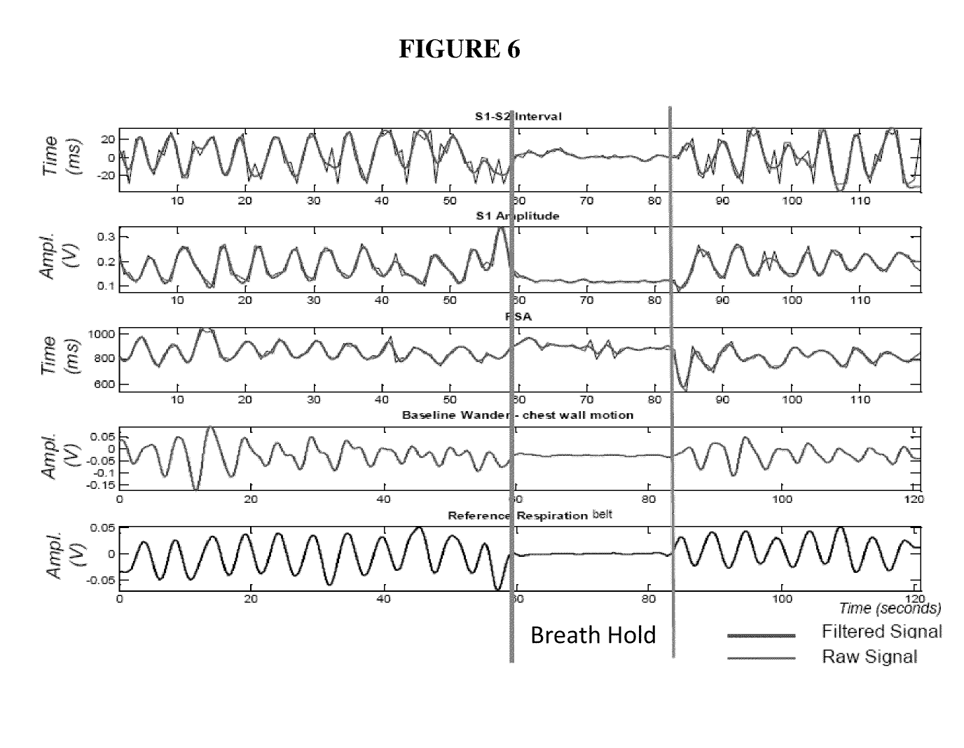

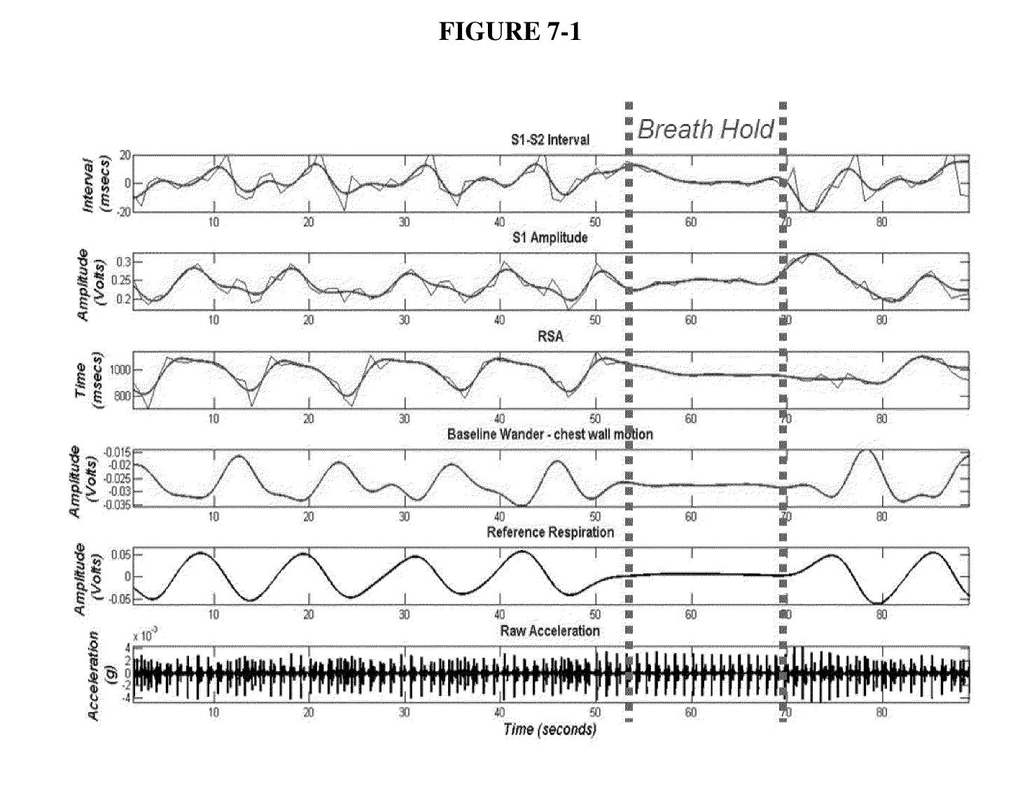

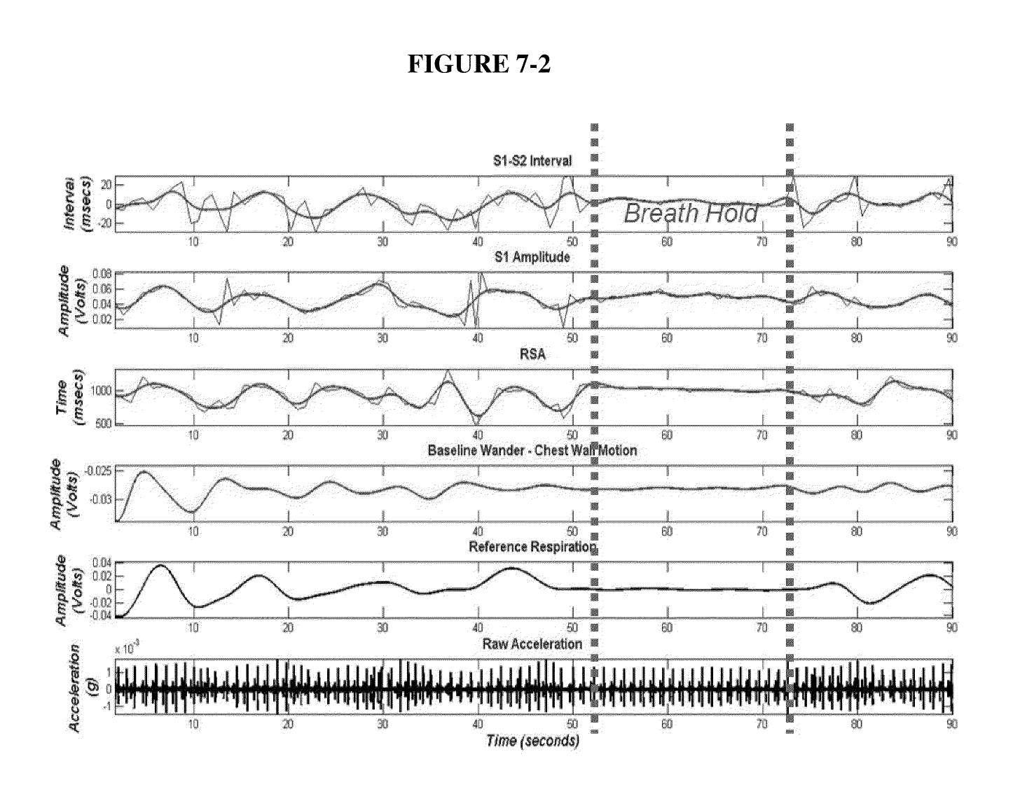

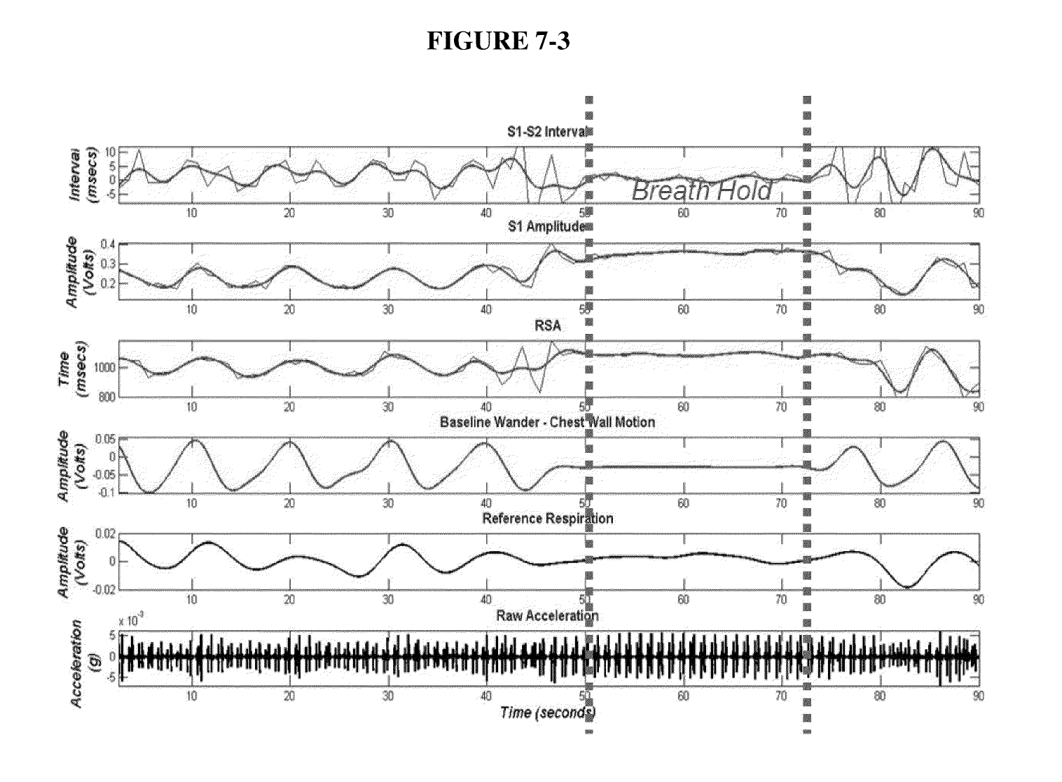

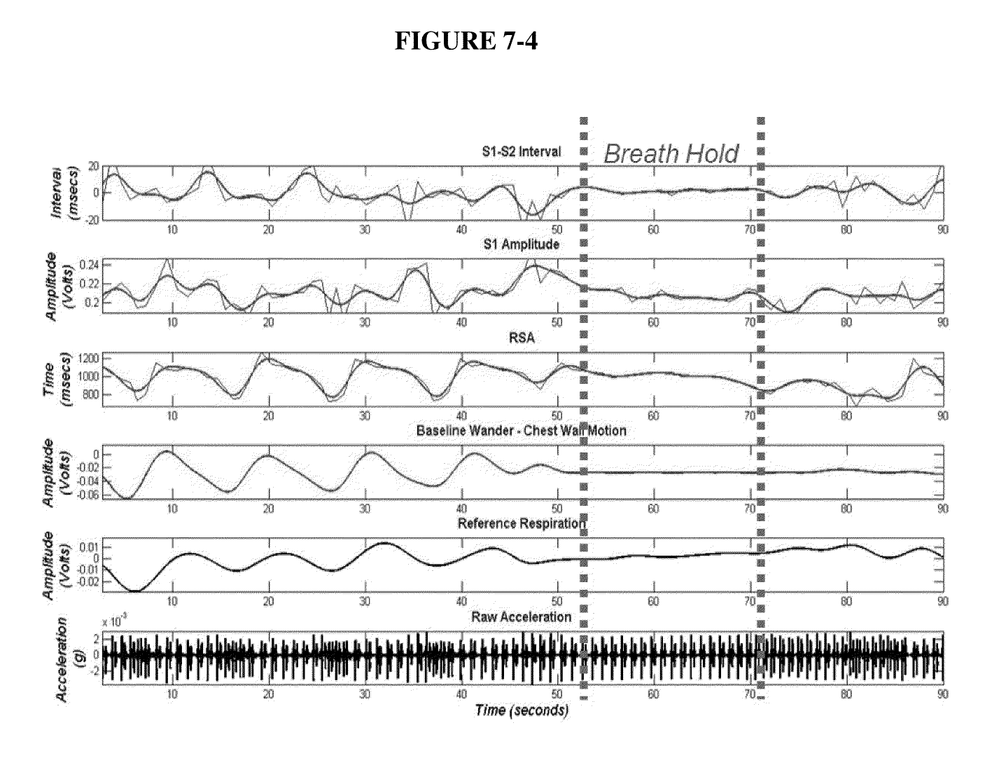

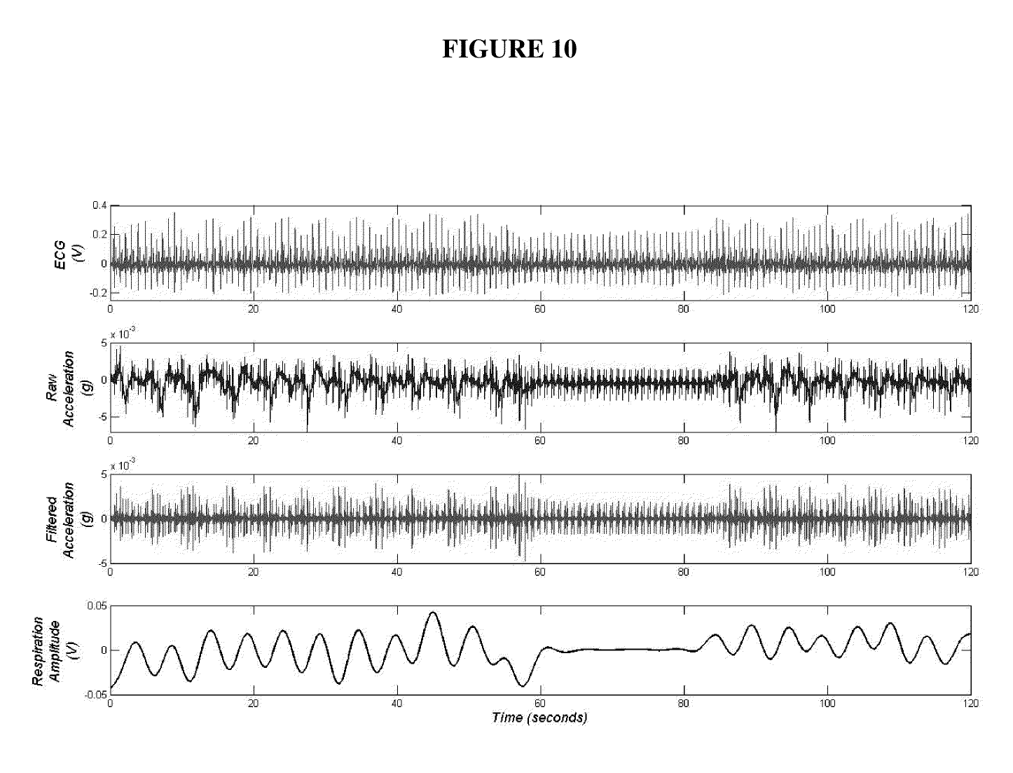

FIGS. 10-1 through 10-4 demonstrate the suitability and reliability of embodiments of the present invention in various postures by illustrating exemplary recordings of chest wall motion, S1 amplitude, S1-S2 interval and S1-S1 interval for an individual in four different positions: supine (FIG. 10-1), prone (FIG. 1-2), on left side (FIG. 10-3) and on right side (FIG. 10-4). These figures show that in the four positions, at least three out of the four extracted respiration waveforms correlate well with the reference respiratory belt. As expected, the prone position leads to a reduced chest wall motion, as shown in FIG. 10.2. However, the three other signals are still able to pick up accurately the respiration pattern, highlighting the robustness of the approach. The chest motion trace could readily be removed from analysis based on simple posture analysis from the accelerometer data. This approach of unobtrusively measuring respiration could be greatly beneficial for applications like sleep apnea and sleep respiration monitoring of an individual by quantifying apneic events and by providing an indication of the type of apneic events that this individual experiences, based on the heart rate and its variability, as evidenced by the respiration traces.

State of Motion

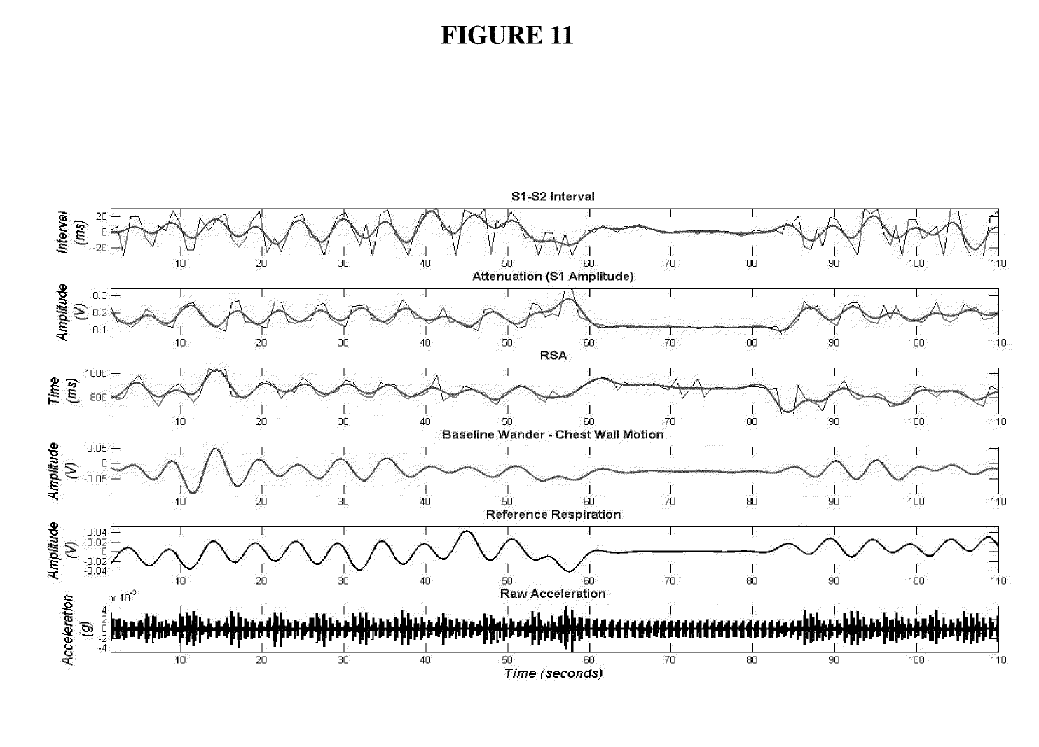

FIG. 11 demonstrates the suitability of embodiments of the present invention in a state of light motion by illustrating chest wall motion, signal amplitude, S1-S2 interval and S1-S1 interval derived from acceleration recordings taken from an individual walking on a treadmill with graded pace, starting with standing rest recording. As outlined in the highlighted section of Table 1, these parameters were analyzed in combination and derived in accordance with embodiments of the present invention, using a chest-worn accelerometer, yielding particularly high correlation coefficients between each other and showing suitability for respiration rate determination in an individual while moving. In contrast, the reference respiration belt was found to be highly sensitive to motion artifacts, yielding poor correlation coefficients between each of the respiration parameters extracted from the accelerometer and the respiration belt, as further illustrated in Table 1.

TABLE 1
Comparative evaluation of respiration waveforms derived from various
acceleration features and from respiration belt in a walking individual.

| Signals | | Correlation Coefficient |
|---|---|---|
| Respiration Belt | S1-S2 Interval | 0.6434 |
| Respiration Belt | RSA | 0.6534 |
| Respiration Belt | Attenuation (S1 Amplitude) | 0.6200 |
| Respiration Belt | Chest Wall Motion | 0.6351 |
| S1-S2 Interval | RSA | 0.8927 |
| RSA | Attenuation | 0.8876 |
| Attenuation | S1-S2 Interval | 0.9014 |

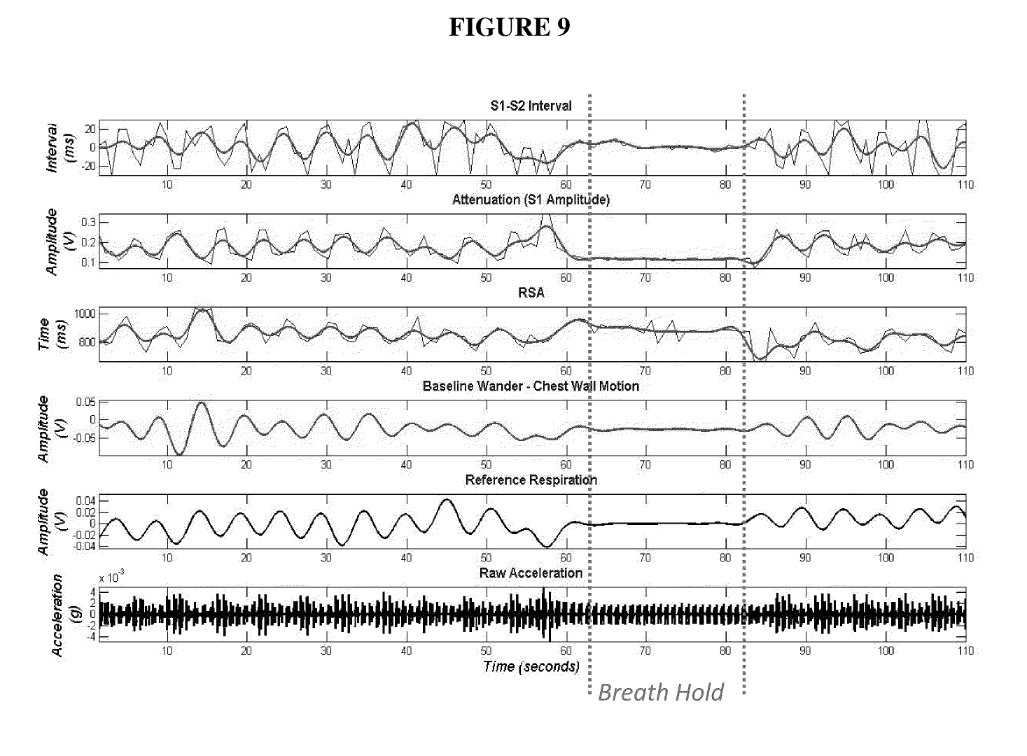

FIG. 9 demonstrates the suitability and reliability of embodiments of the present invention in various states of motion by illustrating chest wall motion, signal amplitude, S1-S2 interval and S1-S1 interval derived from acceleration recordings taken from a seated and resting individual.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### EXPERIMENTAL PROCEDURES

The following methods and materials were used in the examples that are described below.

Computational Analysis

Bland-Altman analysis was used to compare each respiration-dependent parameter derived from the accelerometer to the reference respiration belt in terms of respiration rate (in breaths per minute). A frame-wise Fast Fourier Transform (FFT frame) was computed for 20 second intervals of the four individual respiration-dependent parameters chest wall motion, signal attenuation, S1-S2 interval and S1-S1 interval as well as for the respiration belt signal. This frequency or rate measure was converted to breaths per minute to obtain the respiration rate. For each parameter, the difference between the derived respiration rate and the reference respiration rate (obtained from the respiration belt) was plotted against their mean. The standard deviation and the confidence intervals for each of the parameters were computed assuming a normal distribution. A combined average of the respiration rates derived from the four parameters per 20 second frame was computed and analyzed, as just described.

A moving average across five FFT frames was computed to mitigate false peaks detected by the FFT algorithm. This was computed for each of the four parameters individually, for the combined average of the four and for the respiration belt reference, followed by Bland-Altman analysis, as described.

A correlation-based method of analysis comprised computing a cross-correlation of 10-second windows of each of the individual respiration-dependent parameters chest wall motion, S1 attenuation, S1-S2 interval and S1-S1 interval with respect to the respiration belt reference signal. The accurate values of correlation coefficients were computed after best aligning the two signal frames in phase and equalizing their lengths. The mean correlation coefficient for each parameter across recordings was computed.

### EXAMPLES

The following examples are put forth so as to provide those of ordinary skill in the art with a complete disclosure and description of how to make and use the present invention; they are not intended to limit the scope of what the inventors regard as their invention.

### Example 1

### Monitoring of Respiration Rate in Healthy Individuals Using a Single Chest-Worn Accelerometer

An accelerometer was used in this study as the only sensor, worn by 25 healthy individuals on the chest. The respiration waveforms and rate were determined for these healthy individuals in accordance with various embodiments of the present invention and in comparison to the reference respiration belt.

Experimental Setup

A block diagram of the experimental setup is shown in FIG. 1. A miniature (0.08 gram, 5×5×1.6 mm) triple-axis, low-power, analog-output microelectromechanical systems (MEMS) accelerometer with a sensing element and an integrated circuit interface (LIS3L02AL, STMicroelectronics, Geneva, Switzerland) was taped onto the chest of the recruited individuals, over the 4^{th} rib, about 2-3 inches to the left of the sternum. The seismocardiogram (SCG) chest acceleration signal along the antero-posterior direction (“Z-axis”), orthogonal and in to the plane of the chest, was detected.

The signal was AC coupled and amplified by a gain of 100 and low-pass filtered for anti-aliasing, using a 5-pole Sallen-Key Butterworth filter with a 1 kHz corner frequency. A commercial quad operational amplifier package (LT1014CN, Linear Technology, Milpitas, Calif.) was used for the analog front-end. The accelerometer signal was then sampled at 10 k samples/sec using a data acquisition card (National Instruments, Austin, Tex.) and captured and stored on a: computer using custom software (Matlab, Version 2007b, The Mathworks, Natick, Mass.). The signal was digitally low-pass filtered to 50 Hz before processing it in order to limit sensor noise.

The reference respiration signal was considered the reference signal for this study and was acquired using a piezo-electric respiration belt (DYmedix, Minneapolis, Minn.) fastened around the subject's upper torso. The signal was amplified using a custom analog charge amplifier and low-pass filtered for anti-aliasing. It was also digitally low-pass filtered at 50 Hz before further processing.

Clinical Protocol

Twenty-five individuals, 8 female and 17 male, were recruited at Stanford University, ranging in age from 21 to 58 years; these individuals were unscreened, i.e., they were not specifically recruited because of known conditions of cardiovascular, respiratory or autonomic dysfunction. The procedures for collection of human subject data were in accordance with the 6503 protocol approved by the Stanford IRB.

The recruited individuals were asked to sit in a chair with the accelerometer taped to their chest 2-3 inches to the left of the upper sternum, along the fourth rib. The precise location of placement did neither affect the relative timings of the heart sounds nor the absolute amplitude of the detected signals. The location of sensor placement for the trial was determined empirically in order to get strong amplitudes of S1 as well as S2 across all individuals. In general, the general region around the 4^{th} and 5^{th} rib, a couple of inches to the left of the sternum resulted in a signal with both strong S1 and S2 features.

As a reference, a respiration belt was fastened around each individual's chest. Each individual was asked to breathe at varying respiration rates, including intervals of breathe hold, between one and two minutes each, while the signal components of chest wall motion, signal attenuation, S1-S2 interval and S1-S1 interval were collected and analyzed.

To investigate whether the method and system, as described in this example, were affected by an individual's posture, respiration belt, ECG and accelerometer data were collected for an individual who was asked to lie down in four different positions—supine, prone, left side as well as right side.

To investigate whether the method and system, as described in this example, was affected by an individual's state of motion, i.e. whether it mattered whether an individual was in a still, resting position or moving around, respiration belt, ECG and accelerometer data were collected for an individual who was asked to walk on a treadmill at a graded pace from rest to 1.2 mph at zero incline.

Signal Processing

The chest acceleration signal in the antero-posterior direction corresponding to the SCG, was digitally low-pass filtered at 50 Hz before any further processing. Shown in FIG. 10 is a typical ECG signal along with the raw and baseline-removed acceleration signals and a reference respiration belt signal all acquired simultaneously from a single subject. Different approaches and algorithms, as explained in the following, were used to extract the four individual respiration-dependent parameters as shown in FIG. 11, for the same signal shown in FIG. 10.

Chest Wall Motion

The motion of the chest wall is sensed as a low frequency baseline wander over which rides the higher frequency heart sound signal. This baseline signal is extracted from the composite SCG using a low order Savitzky-Golay polynomial filter. This filter approximates the acceleration signal to capture only the slower varying baseline wander, leaving the higher frequency signal components behind. This filtering approach tries to fit a polynomial of a specified order and frame size (4^{th} order and 2001 points, here, determined empirically) that best matches the acceleration signal in the least squares sense. This signal was low-pass filtered at 0.2 Hz.

A residual signal was obtained by subtracting the polynomial baseline fit from the total acceleration signal and was used for all further processing described below. The residual signal was preprocessed to eliminate spurious peaks that could trigger false S1 detections. Wavelet-based de-noising using a 4^{th} order Daubechies wavelet at a 14^{th} order of decomposition and soft thresholding was used for this preprocessing step.

A folded correlation algorithm was used on the wavelet de-noised signal to further emphasize the S1 and S2 features of SCG signal (Ravindran, 2009). Locations of fiducial S1 peaks were computed using amplitude and timing based thresholding. The residual signal, along with the fiducial S1 locations, was used to compute the other respiration-dependent parameters signal attenuation, S1-S2 interval and S1-S1 interval.

S1 Amplitude

Six metrics of signal amplitude were computed: Root mean square (RMS) power and maximum absolute amplitude of each entire beat as well as of S1 only and of S2 only were evaluated. To this end, the duration of each beat as well as the duration or window of S1 and S2 had to be determined across all acceleration signals. The duration of each beat was found to be no less than 0.7 seconds across all recordings (alternatively, a dynamic range could be used). Every beat was considered to start 0.2 seconds prior to each fiducial S1 peak location so as to leave a buffer window prior to the S1 estimate. This ensured encompassing the entire S1 waveform. The duration of S1 was empirically determined to lie within 20 and 60% of the entire beat and the duration of S2 was determined to be between 65 and 100% of the entire beat. The parameters quantifying amplitude as described above were computed for each beat. Each of these signals was interpolated using a cubic spline interpolation method, re-sampled, and then low-pass filtered at 0.2 Hz to reconstruct the amplitude-derived waveform. As shown in Table 2, the respiration rate derived from the maximum absolute value of the S1 amplitude was found to be closest to that from the respiration belt reference per the Bland-Altman analysis.

TABLE 2
Bland-Altman statistics for several metrics of heart sound amplitude-
derived respiration rates compared with respiration belt-derived rates.

| | | | |
|---|---|---|---|
| | | Standard | 95% Confidence |
| Parameter | Bias | Deviation | Intervals |

| | | | | |
|---|---|---|---|---|
| Power | −0.1340 | 3.0919 | 5.9260 | −6.1941 |
| S1 Power | −0.0873 | 2.9583 | 5.7110 | −5.8856 |
| S2 Power | −0.2635 | 3.3195 | 6.2427 | −6.7697 |
| Amplitude | −0.4934 | 3.3221 | 6.0179 | −7.0048 |
| S1 Amplitude | −0.2215 | 2.6890 | 5.0488 | −5.4919 |
| S2 Amplitude | −0.2345 | 3.6970 | 7.0116 | −7.4805 |

S1-S2 Interval

A different approach was utilized to accurately determine the S1-S2 interval, which varied from beat to beat, and to precisely capture the degree of change. Every beat was matched with its directly preceding beat, whereby those beats were first compressed by 30 milliseconds and then incrementally stretched by varying re-sampling ratios up to a maximum of 30 milliseconds compared to the original beat. The compression or stretch at which a particular beat was found to closest match its directly preceding beat upon cross-correlation was deemed to represent the variation in the S1-S2 interval of that particular beat with respect to its preceding beat. Sixty compression/stretching cycles were used between each adjacent pair of beats and the dilation time in milliseconds corresponding to the best correlation was considered the S1-S2 delay. As described for signal attenuation, the signals were evenly re-sampled, interpolated and low-pass filtered at 0.2 Hz.

S1-S1 (Inter-Heart Beat or Inter-Beat) Interval

The inter-beat interval or interval between consecutive S1 instances was computed by a simple difference between the consecutive fiducial S1 timings. This signal was also evenly re-sampled, interpolated and low-pass filtered at 0.2 Hz, as describe for the signal attenuation and S1-S2 interval.

The described processing was conducted to derive the parameters of chest wall motion, signal attenuation, S1-S2 interval and S1-S1 interval from the data collected from the individual in supine, prone, left-sided or right-sided position.

Under ambulatory conditions, the raw acceleration signal was strongly corrupted by motion. A least-squares based polynomial approximation approach was implemented using the Savitzy-Golay filtering algorithm to track the slow varying motion component (Pandia et al., 2010). A high order (order of 30) polynomial was used to approximate the rapidly varying motion signal and was subtracted from the total acceleration to get a heart-sound residue. Subsequently, those algorithms were used to derive respiration-dependent parameters from the residual heart sound acceleration signal under motion conditions.

Results

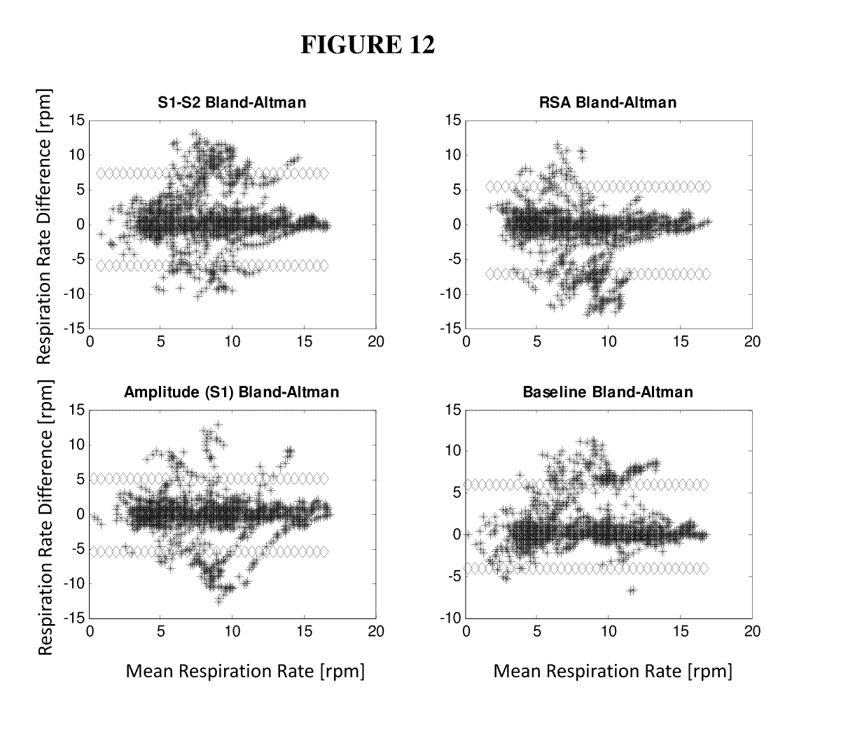

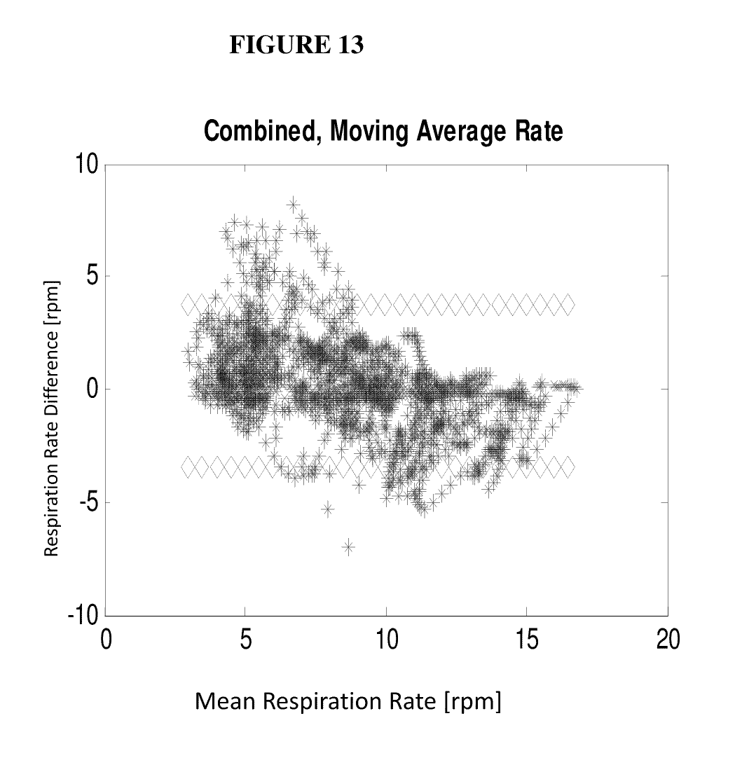

As already mentioned in the experimental procedures section, Bland-Altman analysis was used to compare each respiration-dependent parameter derived from the accelerometer to the respiration belt reference in terms of respiration rate (in breaths per minute). FIG. 12 shows Bland-Altman plots for each individual respiration-dependent parameter chest wall motion, signal attenuation, S1-S2 interval and S1-S1 interval (respiratory sinus arrhythmia, RSA), as computed through the five point moving average relative to the respiration belt reference signal for 23 individuals. The X-axis shows the mean respiration rate (respiration per minute, rpm) over a 15 seconds window, while the Y-axis shows the respiration rate difference between accelerometer and respiration belt; xxx indicates the 95% confidence interval. FIG. 13 shows a Bland-Altman plot for the averaged respiration rate from all four parameters in FIG. 12; xxx indicates again the 95% confidence interval.

Table 3 shows the performance metrics of each of the four individual respiration-dependent parameters, as measured by Bland-Altman analysis as well by the correlation approach combining all four individual parameters.

TABLE 3
Bland-Altman analysis of chest wall motion, signal attenuation,
S1-S1 interval (RSA) and S1-S2 interval individually and in
combination, along with average correlation between acceleration-
derived respiration traces and respiration belt trace.

| | | Standard | 95% Confidence | | Correlation |
|---|---|---|---|---|---|
| Parameter | Bias | Deviation | Intervals | | Coefficient |
| Chest Wall | 1.0051 | 2.5517 | 6.0064 | −3.9962 | 0.8695 |
| Motion | | | | | |
| Attenuation | −0.2215 | 2.6890 | 5.0488 | −5.4919 | 0.8499 |
| RSA | −0.8573 | 3.1698 | 5.3556 | −7.0702 | 0.8479 |
| S1-S2 | 0.6798 | 3.3770 | 7.2987 | −5.9391 | 0.8270 |
| Combined | 0.1713 | 1.8398 | 3.7772 | −3.4346 | |

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Amit et al. (2009). Respiratory modulation of heart sound morphology. Am J Physiol Heart Circ Physiol 296:H796-H805.
Oksenberg et al. (1997). Positional vs Nonpositional Obstructive Sleep Apnea Patients. CHEST 112(3), 629-639.
Ravindran et al. (2009). Real-time, low-complexity, low-memory solution to ECG-based heart rate detection. IEEE Annual International Conference of the Engineering in Medicine and Biology Society, p. 1371-1374.
Grossman et. al. (1993). Respiratory sinus arrhythmia, cardiac vagal tone, and respiration: within- and between-individual relations. Psychophysiology 30, p. 486-495.
Yasuma F. et al. (2004). Respiratory Sinus Arrhythmia-*Why Does the Heartbeat Synchronize With Respiratory Rhythm? CHEST 125(2), p. 683-690.
Pandia K., et al. (2010). Motion artifact cancellation to obtain heart sounds from a single chest-worn accelerometer, IEEE International Conference on Acoustics Speech and Signal Processing (ICASSP), p. 590-593.

What is claimed is:

1. A method of determining an individual's respiration pattern, respiration rate, other cardio-respiratory parameters or variations thereof, the method comprising
sensing
a) mechanical movements of the individual's chest; and/or
b) acoustic waves generated by the individual's heart beats;
processing said mechanical movements and/or acoustic waves to obtain one or more respiration-dependent parameters for subsequent computational analysis of the individual's cardio-respiratory parameters, wherein
one respiration-dependent parameter is a variation in S1-S2 intervals between two consecutive heart beats and another respiration-dependent parameter is a variation in heart sound amplitude.

2. The method of claim 1, wherein the computational analysis comprises
estimating S1-S2 interval variations between a beat and its preceding beat; wherein
beats include both first (S1) and second (S2) heart sounds;
quantifying the similarity between the preceding beat and versions of the beat;
identifying maximum similarity throughout the versions and assigning a corresponding S1-S2 interval variation to the beat based on identified version.

3. The method of claim 2, wherein the heart beat is detected by its first heart sound S1, and wherein assessing a degree of similarity only includes the second sound S2.

4. The method of claim 2, wherein the heart beat is detected by its second heart sound S2, and wherein assessing a degree of similarity only includes the first sound S1.

5. The method of claim 1, wherein an additional respiration-dependent parameter is a variation in S1-S1 intervals.

6. The method of claim 1, wherein an additional respiration-dependent parameter is chest wall motion.

7. The method of claim 1, wherein the computational analysis is carried out with a combined plurality of respiration-dependent parameters.

8. The method of claim 1, wherein the computational analysis is carried out with one respiration-dependent parameter.

9. A method of detecting respiratory disorders in an individual, the method comprising
sensing
a) mechanical movements of the individual's chest; and/or
b) acoustic waves generated by the individual's heart beats;
processing said mechanical movements and/or acoustic waves to obtain one or more respiration-dependent parameters for subsequent computational analysis of the individual's respiration pattern, respiration rate, other cardio-respiratory parameters or variations thereof, wherein one respiration-dependent parameter is a variation in S1-S2 intervals between two consecutive heart beats and another respiration-dependent parameter is a variation in heart sound amplitude.

10. The method of claim 9, wherein the computational analysis comprises
estimating S1-S2 interval variations between a beat and its preceding beat; wherein
beats include both first (S1) and second (S2) heart sounds;
quantifying the similarity between the preceding beat and versions of the beat;
identifying maximum similarity throughout the versions and assigning a corresponding S1-S2 interval variation to the beat based on identified version.

11. The method of claim 9, wherein the heart beat is detected by its first heart sound S1, and wherein assessing a degree of similarity only includes the second sound S2.

12. The method of claim 9, wherein the heart beat is detected by its second heart sound S2, and wherein assessing a degree of similarity only includes the first sound S1.

13. The method of claim 9, wherein an additional respiration-dependent parameter is a variation in S1-S1 intervals.

14. The method of claim 9, wherein an additional respiration-dependent parameter is chest wall motion.

15. The method of claim 9, wherein the computational analysis is carried out with a combined plurality of respiration-dependent parameters.

16. The method of claim 9, wherein the computational analysis is carried out with one respiration-dependent parameter.

17. The method of claim 9, wherein the respiratory disorders are pulmonary hypertension, pulmonary edema, chronic obstructive pulmonary disease, asthma or sleep apnea.

18. A method of detecting an autonomic nervous system disorder in an individual, the method comprising
sensing
a) mechanical movements of the individual's chest; and/or
b) acoustic waves generated by the individual's heart beats;
processing said mechanical movements and/or acoustic waves to obtain one or more respiration-dependent parameters for subsequent computational analysis of the individual's respiration pattern, respiration rate, other cardio-respiratory parameters or variations thereof, wherein
one respiration-dependent parameter is a variation in S1-S2 intervals between two consecutive heart beats and another respiration-dependent parameter is a variation in heart sound amplitude.

19. The method of claim 18, wherein the computational analysis comprises
estimating S1-S2 interval variations between a beat and its preceding beat; wherein
beats include both first (S1) and second (S2) heart sounds;
quantifying the similarity between the preceding beat and versions of the beat;
identifying maximum similarity throughout the versions and assigning a corresponding S1-S2 interval variation to the beat based on identified version.

20. The method of claim 18, wherein the heart beat is detected by its first heart sound S1, and wherein assessing a degree of similarity only includes the second sound S2.

21. The method of claim 18, wherein the heart beat is detected by its second heart sound S2, and wherein assessing a degree of similarity only includes the first sound S1.

22. The method of claim 18, wherein an additional respiration-dependent parameter is a variation in S1-S1 intervals.

23. The method of claim 18, wherein an additional respiration-dependent parameter is chest wall motion.

24. The method of claim 18, wherein the computational analysis is carried out with a combined plurality of respiration-dependent parameters.

25. The method of claim 18, wherein the computational analysis is carried out with one respiration-dependent parameter.

26. The method of claim 18, wherein the autonomic nervous system disorder is syncope.

27. A system of determining an individual's respiration pattern, respiration rate, other cardio-respiratory parameters or variations thereof, the system comprising
at least one sensor for sensing
a) mechanical movements of the individual's chest; and/or
b) acoustic waves generated by the individual's heart beats;
a data acquisition device for receiving signals derived from said mechanical movements and/or acoustic waves;
a processor for processing said signals to obtain one or more respiration-dependent parameters for subsequent computational analysis of the individual's respiration pattern, respiration rate, other cardio-respiratory parameters or variations thereof.

28. The system of claim 27, wherein the computational analysis comprises
estimating S1-S2 interval variations between a beat and its preceding beat; wherein
beats include both first (S1) and second (S2) heart sounds;
quantifying the similarity between the preceding beat and versions of the beat;
identifying maximum similarity throughout the versions and assigning a corresponding S1-S2 interval variation to the beat based on identified version.

29. The method of claim 27, wherein the heart beat is detected by its first heart sound S1, and wherein assessing a degree of similarity only includes the second sound S2.

30. The method of claim 27, wherein the heart beat is detected by its second heart sound S2, and wherein assessing a degree of similarity only includes the first sound S1.

31. The system of claim 27, wherein an additional respiration-dependent parameter is a variation in S1-S1 intervals.

32. The system of claim 27, wherein an additional respiration-dependent parameter is chest wall motion.

33. The system of claim 27, wherein the computational analysis is carried out with a combined plurality of respiration-dependent parameters.

34. The system of claim 27, wherein the computational analysis is carried out with one respiration-dependent parameter.

35. The system of claim 27, wherein the at least one sensor consists of a single-axis accelerometer, a multi-axis accelerometer, a stethoscope, a laser vibrometer or an electromagnetic radar.

36. The system of claim 27, wherein at least one sensor consists of a multi-axis accelerometer, and provides body posture and body motion information.

37. The system of claim 36 for the particular use as a sleep monitoring device.
